SEQUENCE LISTING (4 sequences)

SEQ ID NO: 1 - protein, 18 aa, synthetic construct
  source 1..18: note = The terminal residue X indicates the arbitrary amino acid residue to be determined depending on the conjugation chemistry used for conjugation between peptide and nanoparticle.
  UNSURE 1
XSGGGGPLPSRAPLQHFS

SEQ ID NO: 2 - protein, 13 aa, synthetic construct
  source 1..13: note = The terminal residue X indicates the arbitrary amino acid residue to be determined depending on the conjugation chemistry used for conjugation between peptide and nanoparticle.
  UNSURE 1
XSGGGGPTLLRPY

SEQ ID NO: 3 - DNA, 6885 bp, synthetic construct
  source 1..6885: mol_type = other DNA; note = constructed plasmid SARS-CoV-2-Spike_pUC-GW-Kan
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaattctcaagcttttggaccctcgtacagaagctaatacgactcactatagggaaataagagagaaaagaagagtaagaagaaatataagaccccggcgccgccaccatgtttgtgtttttggtgctccttccgctggtctcctctcaatgcgttaacctgactacacgcacgcaactgccaccggcttatactaattctttcacacgaggtgtgtactatcccgacaaagtgttccgaagctctgttttgcacagtacccaagatctgtttctgccttttttttcaaatgtcacatggtttcacgcgattcatgtgtccgggaccaatggtactaaacgctttgacaacccggttttgccgtttaacgacggcgtatatttcgcgtccacagagaaaagcaacataattagagggtggattttcgggacaacactcgactctaagactcagtcattgttgatcgtaaacaatgctaccaacgtagtaattaaggtttgtgaatttcagttttgcaacgaccccttcctcggggtatattatcacaagaacaataaatcttggatggagtcagaatttcgcgtatattctagtgctaataactgcacctttgaatacgtctcccaaccgttcttgatggatctcgaagggaaacaaggaaactttaagaacctccgagagtttgtcttcaaaaatattgacggatatttcaagatctattccaagcacacgccgatcaaccttgtgcgagaccttccccaaggatttagcgcgctcgagccgcttgtagacctgccgatcggaattaatataacccggttccagaccctcctggctctccatcggtcttacctcacgcctggggattcatcttctggttggactgctggagccgcggcgtattatgttggataccttcagccgcgcacattccttctcaagtacaacgagaacggtaccattactgatgcagtggactgcgctctcgatccactgtccgagacaaagtgtaccctcaaatctttcacggtcgagaaaggcatatatcagacgagcaactttcgggtccaaccgacggaatcaatcgttcgctttcccaacataacgaacctctgccccttcggggaggttttcaacgcaaccaggtttgcgagtgtttatgcatggaatagaaagaggatttcaaattgtgtggctgactacagcgtgttgtacaatagtgccagcttcagtacttttaagtgttacggggttagtcccacaaaactgaatgacctgtgttttactaacgtgtacgcggattccttcgtcatccgcggcgatgaagttcggcaaattgctcccgggcagacgggaaagatcgccgattacaactacaagcttcccgatgatttcactgggtgtgtaattgcctggaatagtaataatctggatagcaaagtaggtggcaactacaattacttgtatcgcctgttccgaaaatccaaccttaaaccgttcgaacgcgacatcagcacagaaatttatcaggctggaagcacaccctgcaacggagtagaagggtttaattgttactttccacttcagagctatgggtttcaacccaccaacggtgtggggtatcaaccgtatagggttgtggtcttgtccttcgagcttctgcatgcgccagctactgtgtgtgggcccaaaaagagtacgaacctcgtgaaaaacaagtgcgtaaactttaactttaatggtctgacaggcacgggggtactcacggagtctaataagaagtttctgccattccagcagtttgggagagacatagccgacactaccgatgccgtgcgcgatccccaaacacttgaaatactggacattacaccatgtagtttcggaggtgtctctgtcattacccccggcaccaacacgtccaaccaggtcgccgtcctgtaccaagacgtaaattgtaccgaggtccctgtagcgatacacgcggaccagctcactccgacatggagagtctactctacggggagtaacgtgttccagacacgcgctggctgtcttataggcgccgaacatgtcaacaattcatacgagtgtgatattcctataggggcgggtatttgcgcgtcataccagacccaaacaaactctccaagacgggctcgctcagtggccagccagagcataatcgcctatacaatgtcacttggggccgagaactccgttgcgtacagtaataattcaatagccattccaacgaacttcacgatatcagttactacggagatccttccagtcagtatgaccaaaacgtctgtcgattgcacgatgtacatctgcggggactccacggaatgcagtaacttgcttctgcaatacgggtctttttgcacccaactcaatcgcgcactgaccggtattgccgtggaacaagacaagaacacccaggaggtcttcgcccaagtaaagcagatttataaaactcctcccattaaagactttgggggttttaacttttcacaaatacttcccgatccatctaagccaagcaaacgaagctttatagaagacctccttttcaataaagttacgcttgctgacgcaggattcattaagcaatacggcgattgtctgggagatattgcagcgagggatcttatttgtgcgcaaaagtttaacggacttaccgtgcttccccctttgcttacggacgaaatgatcgcgcaatacacgagcgcgctgcttgctggtacaattacctcaggatggacttttggtgcgggagctgcccttcagatcccgttcgctatgcaaatggcgtatcgcttcaatggtattggtgtgactcaaaacgtattgtatgaaaaccagaagctcatagcaaatcagttcaacagtgccataggtaagattcaggactctttgagctccactgcatctgcccttgggaaactgcaagatgtggtgaaccaaaatgctcaggcccttaacactttggtgaagcaactgtccagcaatttcggtgctatttcaagtgtccttaatgatatcctctccaggctcgatccaccggaggcagaagtccagatagatcgccttattaccggccgcctccaatccttgcaaacgtatgtcacccagcaattgatccgcgcagctgagatacgggcttccgcgaatctggcggcaactaaaatgagcgaatgtgtcctcgggcaaagtaaacgcgtggacttttgcggcaagggttaccacctgatgtcttttccgcaatccgcacctcacggggtggtctttctccacgttacctacgtacccgcccaggaaaagaacttcactactgctccggccatctgccatgatgggaaagcgcactttcccagggaaggtgtttttgtgtccaacgggacccattggttcgtgacgcaacggaacttttatgagccgcagattataacgaccgacaacactttcgtaagtggtaattgtgatgttgttatcggaatcgttaataacacggtctatgaccccctccagccggagcttgactctttcaaagaagagcttgataaatattttaagaaccacaccagtccggatgttgaccttggcgacatcagtgggattaacgcttccgtagtgaatatccagaaggagatagatcgattgaacgaagtggcaaagaacctcaatgagtctttgatcgacctgcaggagctcggtaagtatgagcagtatatcaaatggccctggtacatttggctggggtttattgctggcctcattgctatcgttatggtaaccataatgctttgttgtatgacgtcctgctgcagttgcctgaaagggtgctgcagttgtggctcttgttgtaaatttgacgaagacgattctgaacccgtacttaaaggagtgaagctgcactacacgtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagtcgacttggctgctgccaccgctgagcaataactagcataaccccttggggcctctaaacgggtcttgaggggttttttgctgaaaggaggaactatatccggatgcggccgcatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 4 - DNA, 4975 bp, synthetic construct
  source 1..4975: mol_type = other DNA; note = constructed plasmid Flu-HA_pUC-GW-Kan
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaattctcaagcttttggaccctcgtacagaagctaatacgactcactatagggagagcggccgctttttcagcaagattaagcccagggcagagccatctattgcttacatttgcttctgacacaactgtgttcactagcaacctcaaacagacaccgccaccatgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggarattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggrtcragaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttgataatagagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagtcgacttggctgctgccaccgctgagcaataactagcataaccccttggggcctctaaacgggtcttgaggggttttttgctgaaaggaggaactatatccggatgcggccgcatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

(19) United States
(12) Patent Application Publication
(10) Pub. No.: US 2024/0390483 A1
(43) Pub. Date: Nov. 28, 2024

(54) MUCOSAL MESSENGER RNA VACCINE

(71) Applicant: iNtRON Biotechnology, Inc., Gyeonggi-do (KR)

(72) Inventors: Seong Jun YOON, Seoul (KR); Soo Youn JUN, Seoul (KR); Ji Sung PARK, Gyeonggi-do (KR); Sang Hyeon KANG, Seoul (KR)

(21) Appl. No.: 18/691,448

(22) PCT Filed: Sep. 9, 2022
(86) PCT No.: PCT/IB2022/058528

§ 371 Date: Mar. 12, 2024

Related U.S. Application Data
(60) Provisional application No. 63/243,086, filed on Sep. 10, 2021.

(51) Int. Cl. A61K 39/215; A61K 39/00; A61K 39/145; A61K 47/64; A61K 47/69; A61P 37/04

(52) U.S. Cl. CPC A61K 39/215; A61K 39/145; A61K 47/64; A61K 47/6931; A61P 37/04; A61K 2039/53

(57) ABSTRACT

The present invention is related to a mucosal mRNA vaccine. More specifically, the present invention relates to novel mucosal mRNA vaccine based on a peptide-conjugated, mRNA loaded nanoparticle with enhanced vaccine efficacy, and a method of preparing the same.

This application claims priority to U.S. Provisional Patent Application No. 63/243,086, filed on Sep. 10, 2021, which is incorporated by reference for all purposes as if fully set forth herein.

### BACKGROUND OF THE INVENTION

### Field of the Invention

The present invention is related to a mucosal messenger RNA vaccine. More specifically, the present invention relates to novel mucosal messenger RNA vaccine based on a peptide-conjugated, messenger RNA loaded nanoparticle with enhanced vaccine efficacy, and a method of preparing the same.

### Discussion of the Related Art

Infectious disease caused by Severe Acute Respiratory Syndrome Coronavirus 2 (SARS-COV-2) has recently become a major problem worldwide. As a result, interest in vaccines has increased more than ever.

A vaccine is a biological preparation that provides active acquired immunity to a particular infectious disease. A vaccine typically contains an agent that resembles a disease-causing microorganism and is often made from weakened or killed forms of the microbe, its toxins, or one of its surface proteins. The agent in vaccines stimulates the body's immune system to recognize the agent as a threat, destroy it, and to further recognize and destroy any of the microorganisms associated with that agent that it may encounter in the future. Vaccines can be prophylactic (to prevent or ameliorate the effects of a future infection by a pathogen), or therapeutic (to treat a disease that has already occurred, such as cancer). The administration of vaccines is called vaccination. Vaccination is the most effective method of preventing infectious diseases.

Conventional vaccine approaches, such as live attenuated and inactivated pathogens and subunit vaccines, provide durable protection against a variety of dangerous diseases. Despite this success, there remain major hurdles to vaccine development against a variety of infectious pathogens, especially those better able to evade the adaptive immune response. Moreover, for most emerging virus vaccines, the main obstacle is not the effectiveness of conventional approaches but the need for more rapid development and large-scale deployment. Finally, conventional vaccine approaches may not be applicable to non-infectious diseases, such as cancer. The development of more potent and versatile vaccine platforms is therefore urgently needed.

Nucleic acid therapeutics have emerged as promising alternatives to conventional vaccine approaches. The first report of the successful use of in vitro transcribed (IVT) messenger RNA (mRNA) in animals was published in 1990, when reporter gene mRNAs were injected into mice and protein production was detected. A subsequent study in 1992 demonstrated that administration of vasopressin-encoding mRNA in the hypothalamus could elicit a physiological response in rats. However, these early promising results did not lead to substantial investment in developing mRNA therapeutics, largely owing to concerns associated with mRNA instability, high innate immunogenicity and inefficient in vivo delivery. Instead, the field pursued DNA-based and protein-based therapeutic approaches.

Over the past decade, major technological innovation and research investment have enabled mRNA to become a promising therapeutic tool in the fields of vaccine development. The use of mRNA has several beneficial features over subunit, killed and live attenuated virus, as well as DNA-based vaccines. First, safety: as mRNA is a non-infectious, non-integrating platform, there is no potential risk of infection or insertional mutagenesis. In contrast to DNA vaccines, the FDA does not consider non-replicating mRNA vaccines gene therapies. Additionally, mRNA is degraded by normal cellular processes, and its in vivo half-life can be regulated through the use of various modifications and delivery methods. The inherent immunogenicity of the mRNA can be down-modulated to further increase the safety profile. Second, efficacy: various modifications make mRNA more stable and highly translatable. Efficient in vivo delivery can be achieved by formulating mRNA into carrier molecules, allowing rapid uptake and expression in the cytoplasm of the target cells. mRNA is the minimal genetic vector; therefore, anti-vector immunity is avoided, and mRNA vaccines can be administered repeatedly. Third, production: mRNA vaccines have the potential for rapid, inexpensive and scalable manufacturing, mainly owing to the high yields of in vitro transcription reactions. The mRNA vaccine field is developing extremely rapidly. But there are still many improvements required. In particular, the development of mRNA vaccine technology in the form of mucosal vaccine is still in its early stages, and rapid technology development is needed.

Administration of vaccines via mucosal routes can provide significant advantages over systemic delivery. The first is that administration is easy. No sterile needles or trained personnel are required. Second, the administration of vaccines through the mucous membrane can cause a strong mucosal immune response, which can provide benefits in terms of efficacy. The induction of mucosal immunity is a highly desirable feature in vaccines because it can provide a first line of defense against many types of infections invading through the mucosal surface. For this reason, interest in mucosal vaccine is increasing significantly. The administration routes of mucosal vaccines may represent the alimentary tract, the respiratory tract, the urogenital tract, and the eye. Epithelia in these sites generally have a mucosa as a defensive structure. The mucous membrane acts as a barrier to microorganism or particle invasion.

Development of mucosal mRNA vaccine is required because they can provide significant benefits in many respects: mucosal vaccination with mRNA vaccine can stimulate both systemic and mucosal immunity and has the advantage of being a non-invasive procedure suitable for immunization of large populations. However, mucosal vaccination with mRNA vaccine is hampered by the lack of efficient delivery of the mRNA encoding antigen. If an mRNA vaccine with improved mucosa-penetration efficiency is developed, it can further improve the effectiveness of the mucosal mRNA vaccine. Efficient delivery of mucosal mRNA vaccines will be key for their success and translation to the clinic.

### SUMMARY OF THE INVENTION

Accordingly, the present invention has been made keeping in mind the problems encountered in the related art and is intended to solve such problems.

In one embodiment, the present invention discloses a peptide-conjugated, mRNA loaded nanoparticle having enhanced vaccine efficacy. The peptide-conjugated, mRNA loaded nanoparticle of the present invention is decorated with a peptide having the amino acid sequence as set forth in SEQ ID NO: 1, a peptide having the amino acid sequence as set forth in SEQ ID NO: 2, or both a peptide having the amino acid sequence as set forth in SEQ ID NO: 1 and a peptide having the amino acid sequence as set forth in SEQ ID NO: 2.The terminal residue “X” of the SEQ ID NO: 1 and SEQ ID NO: 2 indicates the arbitrary amino acid residue to be determined depending on the conjugation chemistry used for conjugation between peptide and nanoparticle.

In another embodiment, the present invention provides a method of preparing an mRNA vaccine having a peptide-conjugated, mRNA loaded nanoparticle with enhanced vaccine efficacy. The peptide-conjugated, mRNA loaded nanoparticle is decorated with a peptide having the amino acid sequence as set forth in SEQ ID NO: 1, a peptide having the amino acid sequence as set forth in SEQ ID NO: 2, or both a peptide having the amino acid sequence as set forth in SEQ ID NO: 1 and a peptide having the amino acid sequence as set forth in SEQ ID NO: 2. The method includes: preparation of mRNA; formation of mRNA loaded nanoparticles; and conjugation of peptide to the preformed, mRNA loaded nanoparticles. The terminal residue “X” of the SEQ ID NO: 1 and SEQ ID NO: 2 indicates the arbitrary amino acid residue to be determined depending on the conjugation chemistry used for conjugation between peptide and nanoparticle.

It is to be understood that both the foregoing general description and the following detailed description are exemplary and explanatory and are intended to provide further explanation of the invention as claimed.

### ADVANTAGEOUS EFFECTS OF INVENTION

In accordance with the present invention, the mucosal mRNA vaccine of the present invention provides improved vaccine efficiency compared to conventional mucosal mRNA vaccine based on conventional nanoparticle without conjugation of peptide.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a map of in vitro transcription plasmid encoding antigen (prefusion stabilized spike protein) of SARS-COV-2.

FIG. 2 is a map of in vitro transcription plasmid encoding antigen (stalk domain of hemagglutinin) of influenza A H1N1 virus.

FIG. 3 is the results of immune response elicited vaccination with the SARS-CoV-2 mucosal mRNA vaccine.

FIG. 4 is the results of immune response elicited vaccination with the influenza A H1N1 virus mucosal mRNA vaccine.

### DETAILED DESCRIPTION OF THE ILLUSTRATED EMBODIMENTS

Reference will now be made in detail to embodiments of the present invention, example of which is illustrated in the accompanying drawings.

Thus, in accordance with one aspect of the present invention, a peptide-conjugated, mRNA loaded nanoparticle is coated with at least one peptide selected from the group of a peptide having the amino acid sequence as set forth in SEQ ID NO: 1 (hereafter P-1) and a peptide having the amino acid sequence as set forth in SEQ ID NO: 2 (hereafter P-2). Thus, the exposure of peptide on the surface of the mRNA loaded nanoparticles may expose peptide P-1 only, peptide P-2 only, or both peptides P-1 and P-2. The terminal residue “X” of the SEQ ID NO: 1 and SEQ ID NO: 2 indicates the arbitrary amino acid residue to be determined depending on the conjugation chemistry used for conjugation between peptide and nanoparticle.

It is not clear yet, but it is assumed that the enhancement effect of the mucosal mRNA vaccine of the present invention may be due to enhanced mucosa-penetration efficiency attributed to the selective binding of the peptide attached to the surface of nanoparticles to the M cell (Microfold cell) surface or the components in the mucosa. M cells represent a potential portal for mucosal drug and vaccine delivery since they possess a high transcytotic capacity and are able to transport a broad range of materials including particulates.

We hypothesized that exposed peptide having the amino acid sequence as set forth in SEQ ID NO: 1 or SEQ ID NO: 2 on the surface of mRNA loaded nanoparticles could aid enhancing the mucosa-penetration efficiency and also result in more improved efficacy of mucosal mRNA vaccine. We demonstrated that our mucosal mRNA vaccine based on the mRNA loaded nanoparticles with modified peptide having the amino acid sequence as set forth in SEQ ID NO: 1 or SEQ ID NO: 2 provided a more improved mucosal mRNA vaccine efficacy, in comparison to the conventional mRNA loaded nanoparticles without surface modification with peptide.

The peptides P-1 and P-2 may explicitly and partially be modified by those skilled in the art using the disclosed contents. The said modification includes partial substitution, addition and deletion of one or more amino acids in the amino acid sequences. That being said, it is most desirable to apply correspondingly the amino acid sequences set forth in SEQ ID NO: 1 and SEQ ID NO: 2 as disclosed in the present invention, because the sequences provided in this invention were designed to have the enhanced mucosa-penetration efficiency based on the applicants' expertise and experience. Specifically, the amino acids sequences provided in this invention were designed considering favorable size, immunogenicity, etc. The terminal residue “X” of the SEQ ID NO: 1 and SEQ ID NO: 2 indicates the arbitrary amino acid residue to be determined depending on the conjugation chemistry used for conjugation between peptide and nanoparticle.

The nanoparticle of the present invention may synthetic, natural lipid or polymeric nanoparticle, but not limited thereto. In general, lipid nanoparticles may be made of cationic lipid, sterol, phospholipids, and PEG lipid. The cationic lipid is selected from the group comprising N-[1-(2,3-dioleyloxy)propyl]-N,N,N-trimethylammonium (DOTMA), 1,2-dioleoyl-3-dimethylammonium-propane (DOTAP), 2,3-dioleyloxy-N-[2(spermine-carboxamido)ethyl]-N,N-dimethyl-1-propanaminium) (DOSPA), 1,2-dilinoleyloxy-N,N-dimethyl-3-aminopropane (DLin-DMA), 2,2-dilinoleyl-4-(2-dimethylaminoethyl)-[1,3]-dioxolane (DLin-KC2-DMA), O-(Z,Z,Z,Z-heptatriaconta-6,9,26,29-tetraen-19-yl)-4-(N,N-dimethylamino) (DLin-MC3-DMA), 3,6-bis({4-[bis(2-hydroxydodecyl)amino]butyl})piperazine-2,5-dione (cKK-E12), N,N-dimethyl-N-octadecyloctadecan-1-aminium chloride (DODAC), didodecyldimethylammonium bromide (DDAB), dioctadecylamidoglycylspermine (DOGS), 1,2-dioleoyl-3-dimethylammonium-propane (DODAP), 1,2-dioleyloxy-N,N-dimethyl-3-aminopropane (DODMA), 1,2-dilinolenyloxy-N,N-dimethyl-3-aminopropane (DLenDMA), 2,3-Dilinoleoyloxy-N,N-dimethylpropylamine (DLinDAP), 1,2-dilinoleoylcarbamyl-3-dimethylaminopropane (DLinCDAP), 2,2-dilinoleyl-4-dimethylaminoethyl-[1,3]-dioxolane (DLin-K-XTC2-DMA), (15Z,18Z)-N,N-dimethyl-6-(9Z,12Z)-octadeca-9,12-dien-1-yl)tetracosa-5,18-dien-1-amine (HGT5000). The sterol is selected from the group comprising cholesterol, ergosterol, campesterol, oxysterol, antrosterol, desmosterol, nicasterol, sitosterol and stigmasterol; preferably cholesterol. Suitable phospholipids within the context of the invention can be selected from the group comprising: 1,2-dioleoyl-sn-glycero-3-phosphoethanolamine (DOPE), 1,2-dioleoyl-sn-glycero-3-phosphocholine (DOPC), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. The term “PEG lipid” or alternatively “PEGylated lipid” is meant to be any suitable lipid modified with a PEG (polyethylene glycol) group. In a particular embodiment, said PEG lipid is selected from the list comprising: PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. More specific examples of such PEG lipids encompass C14-PEG2000 (1,2-dimyristoyl-rac-glycerol, methoxypolyethylene glycol-2000 (DMG-PEG2000) and C18-PEG5000 (1,2-distearoyl-rac-glycerol, methoxypolyethylene glycol-5000 (DSG-PEG5000). The polymeric nanoparticle may be made using the polymer selected from the group consisting of, but not limited to, branched polyamines, branched polyethers, branched polyesters, branched polyureas, branched polysulfones, branched polyacrylic acids, branched polyacrylonitriles, branched polylysines, branched poly-beta-amino esters, branched polyarginines, branched polyaspartamides, branched polyethyleneimines, dendritic PAMAM macromolecular polymers, and combinations thereof, with degrees of branching including, but not limited to, three arms, four arms, six arms, eight arms, sixteen arms, and combinations thereof, with side chain modifications including, but not limited to, diethyltriamine, triethylenetetramine, imidazole, and combinations thereof.

Accordingly, examples of such nanoparticles, but are not limited to, LNPs composed of DSPC, Cholesterol, DOTMA, and DMG-PEG2000, LNPs composed of DSPC, Cholesterol, DOTAP, and DMG-PEG2000, LNPs composed of DSPC, Cholesterol, DLin-KC2-DMA, and DMG-PEG2000, LNPs composed of DSPC, Cholesterol, DLin-MC3-DMA, and DMG-PEG2000, LNPs composed of DSPC, Cholesterol, DLin-DMA, and DMG-PEG2000, LNPs composed of DPPC, Cholesterol, DLin-DMA, and DMG-PEG2000, LNPs composed of DOPE, Cholesterol, DLin-DMA, and DMG-PEG2000, LNPs composed of DOPG, Cholesterol, DLin-DMA, and DMG-PEG2000, LNPs composed of DSPC, Cholesterol, DLin-DMA, and 1,2-distearoyl-sn-glycero-3-phosphoethanolamine-N-[amino(polyethylene glycol)-2000] (DSPE-PEG2000), LNPs composed of DPPC, Cholesterol, DLin-DMA, and DSPE-PEG2000, liposomes, polymeric poly (ethylene glycol) (PEG)-poly (lactic-co-glycolic acid) (PLGA) nanoparticles, PLGA-4-arm-PEG polymeric nanoparticles, PLGA-PEG-PLGA nanoparticles, polymeric Poly (vinyl alcohol) (PVA)-PLGA nanoparticles and the like.

The conjugation of peptide to nanoparticle in the present invention was accomplished via covalent coupling between cysteine of peptide and maleimide attached to the head group of the phospholipid. However, it is self-evident that a variety of chemistry can be applied, but not limited thereto. Accordingly, examples of such covalent coupling, but are not limited to, amide bond, thioether bond, carbamate ester bond, carboxylic acid ester bond, hydrazone bond and the like. Depending on the selected coupling chemistry, appropriate functional group should be incorporated to any component of nanoparticle such as zwitterionic lipid components, PEG, or cholesterol, and appropriate terminal amino acid residue should be selected, which is easy to those skilled in the art.

Also, in general, mRNA molecules may encode a specific antigen or any other therapeutically active protein, suitable for a specific therapy, typically do not show a significant or even no immunostimulatory property.

In accordance with another aspect of the present invention, the present invention provides a peptide-conjugated, mRNA loaded nanoparticle enclosing mRNA encoding antigen (prefusion stabilized spike protein) of SARS-COV-2 or antigen (stalk domain of hemagglutinin; HA) of influenza A H1N1 virus. But, the present invention may be applicable to prepare a peptide-conjugated, mRNA loaded nanoparticle enclosing mRNA encoding various bacterial or viral antigens, or various disease-associated antigens such as cancer-associated antigen. In addition, with respect to viral antigens, the viral antigens of the present invention may be derived from various categories of virus including respiratory virus, not limited to.

Also, in accordance with another aspect of the present invention, the present invention provides a mucosal mRNA vaccine used for prophylactic (to prevent or ameliorate the effects of a future infection by a pathogen) purpose. But, the present invention may be applicable to a mucosal mRNA vaccine used for therapeutic (to treat a disease that has already occurred, such as cancer) purpose.

The mRNA vaccine of the present invention may be used to induce antigen-specific immune response (e.g., a T cell response or a B cell response), for example, in the mucosal tissues of a subject. Depending on the content of the mRNA vaccine, it may be used to immunize a subject against a pathogen (e.g., a mucosal pathogen). The mRNA vaccine of the present invention may also be used to treat an infection (e.g., a mucosal infection or bacteria) in a subject.

The mucosal mRNA vaccine of the present invention may be administered by mucosal administration. As used herein, the term “mucosal administration” refers to a dosage form given via the mucosa. Accordingly, examples of such routes of mucosal administration include, but are not limited to, nasal cavity administration (nasal administration), buccal administration, intravaginal administration, upper airway administration, alveolar administration and the like. In addition, the mRNA vaccine of the present invention may be administered through intravenous, intramuscular, or subcutaneous administration like conventional vaccines.

Also, the appropriate dosage for administering the foregoing mucosal mRNA vaccine varies with such factors as formulation, administration, age, body weight, severity of symptoms, foods, administration time, administration routes, discharge speed and susceptibility in response. Usually, skilled physicians may decide and prescribe with ease the dosage effective for desired vaccination.

As used herein, the term “mucosa” refers to a mucous membrane (rich in mucous glands) that lines body passages and cavities which communicate directly or indirectly with the exterior (as the alimentary, respiratory, and genitourinary tracts), that functions in protection, support, nutrient absorption, and secretion of mucus, enzymes, and salts, and that consists of a deep vascular connective-tissue stroma which in many parts of the alimentary canal contains a thin but definite layer of nonstriated muscle and a superficial epithelium which has an underlying basement membrane and varies in kind and thickness but is always soft and smooth and kept lubricated by the secretions of the cells and numerous glands embedded in the membrane. In exemplary embodiments, the mucosa is the mucous membrane of the nose, vagina, rectum, mouth or intestines.

In this description, the term “coated”, “decorated” or “attached” was used in the same sense, indicating “covalently conjugated onto the surface of nanoparticle”.

As used herein, the term “administer”, “administering”, or “administration” refers to implanting, applying, absorbing, or inhaling, not limited to.

As used herein, the term “subject” refers to an individual organism, for example, an individual mammal. In some embodiments, the subject is a human. In another embodiments, the subject is a non-human mammal.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1: Construction and preparation of plasmids to be used for mRNA synthesis

Example 1-1: Plasmid encoding SARS-COV-2 antigen

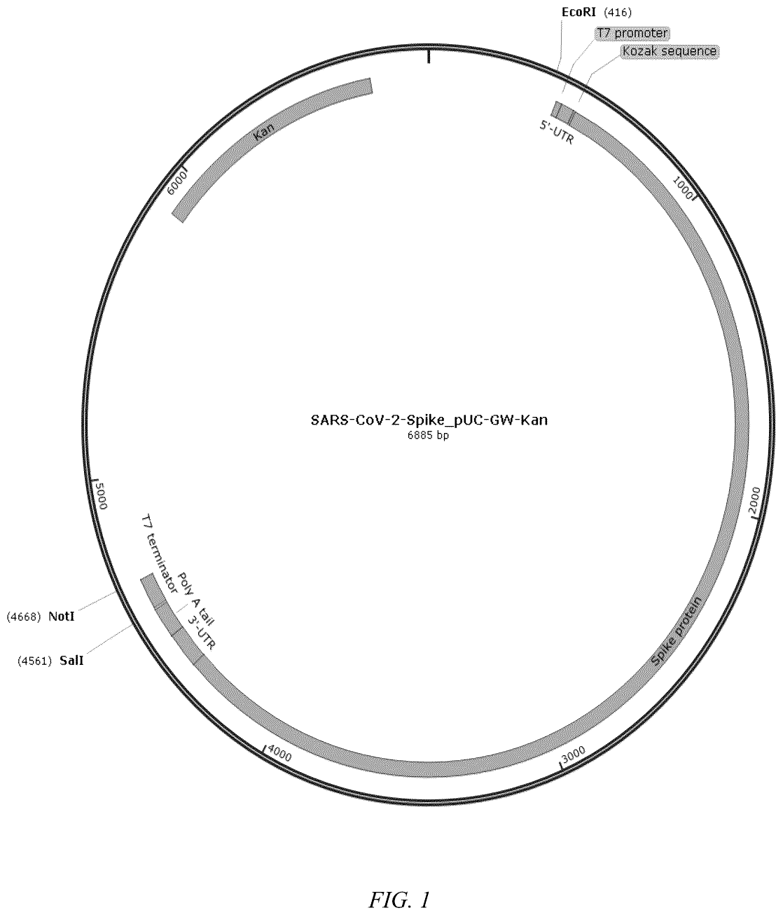

In this Example, in vitro transcription plasmid encoding the sequence-optimized antigen sequence of SARS-COV-2 (a prefusion stabilized spike protein after introducing two proline substitutions in the S2 subunit) including 5′ untranslated region (UTR), 3′ UTR sequences and polyA tail was constructed using conventional gene synthesis and conventional cloning method. The map of constructed plasmid (SARS-COV-2-Spike_pUC-GW-Kan) is shown in FIG. 1, and the nucleotide sequence of plasmid is shown in SEQ ID NO: 3.

The constructed plasmid was used for transformation of E. coli DH5 alpha according to standard procedures to construct plasmid producing strain. The plasmid DNA was prepared from appropriate amount of cultures of the constructed plasmid producing strain by a conventional method. The run-off transcription template was then prepared by incubation of the prepared plasmid with the appropriate restriction enzyme (Sal I). After linearization, the linearized plasmid was purified by typical phenol/chloroform extraction method and used as a template DNA for the mRNA synthesis.

Example 1-2: Plasmid encoding influenza A H1N1 virus antigen

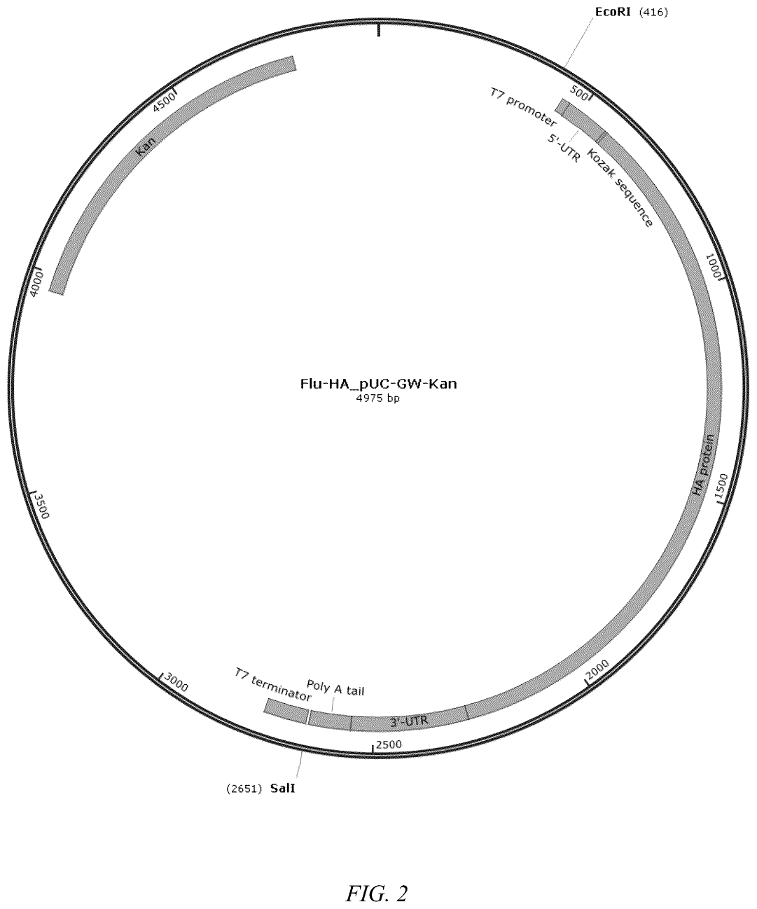

In this Example, in vitro transcription plasmid encoding the sequence-optimized antigen sequence of influenza A H1N1 virus (stalk domain of HA) including the sequences of 5′ and 3′ UTRs originating from human beta-globulin and polyA tail was constructed using conventional gene synthesis and conventional cloning method. The map of constructed plasmid (Flu-HA_pUC-GW-Kan) is shown in FIG. 2, and the nucleotide sequence of plasmid is shown in SEQ ID NO: 4.

The constructed plasmid was used for transformation of E. coli DH5 alpha according to standard procedures to construct plasmid producing strain. The plasmid DNA was prepared from appropriate amount of cultures of the constructed plasmid producing strain by a conventional method. The run-off transcription template was then prepared by incubation of the prepared plasmid with the appropriate restriction enzyme (Sal I). After linearization, the linearized plasmid was purified by typical phenol/chloroform extraction method and used as a template DNA for the mRNA synthesis.

### Example 2: Preparation of mRNA

Example 2-1: mRNA encoding SARS-COV-2 antigen

The in vitro transcription reaction utilizes a custom mixture of nucleotide

triphosphates (NTPs). The NTPs may comprise chemically modified NTPs, or a mixture of natural and chemically modified NTPs, or natural NTPs. In this Example, 1-methylpseudouridine-5′-triphosphate was used instead of uridine triphosphate (UTP).

A typical in vitro transcription reaction includes the following:
1) Template DNA 1.0 μg
2) 5× transcription buffer 4.0 μl
   (600 mM HEPES-KOH (pH 7.5), 180 mM MgCl₂, 325 mM DTT, 15 mM Spermidine)
3) Bovine serum albumin 2.0 μl
4) NTPs (100 mM each) 8.0 μl
5) RNase inhibitor 20 Units
6) T7 RNA polymerase 100 Units
7) DEPC-treated water up to 20.0 ul

Using the prepared mixture for in vitro transcription reaction, incubation was performed at 37° C. for 2 hours. The crude IVT mixture may be stored at 4° C. overnight for cleanup the next day. The crude IVT mixture was treated with 5 Units of RNase-free DNase I to digest the template DNA. After 30 minutes of incubation at 37° C., the mRNA was purified using Monarch® RNA Cleanup Kit (New England Biolabs) according to the manufacturer's instructions. Following the cleanup, the mRNA was quantified using the NanoDrop (Thermo Fisher Scientific) and analyzed by agarose gel electrophoresis to confirm the mRNA is the proper size and that no degradation of the mRNA has occurred.

One-step capping and 2′-O-methylation capping of uncapped mRNA was performed according to the manufacturers' instructions (New England Biolabs). First, uncapped mRNA and DEPC-treated water were mixed in a final volume of 13.5 μl. The mixture is incubated at 65° C. for 5 minutes to denature mRNA, and then was transferred immediately to ice. The protocol then involves the mixing of 10× capping buffer (2.0 μl); 10 mM GTP (1.0 μl); 4 mM S-adenosyl methionine (SAM, 1.0 μl); RNase inhibitor (20 Units); Vaccinia capping enzyme (10 Units); 2′-O-methyltransferase (50 Units) and incubated at 37° C. for 1 hour. The capped mRNA was purified using Monarch® RNA Cleanup Kit (New England Biolabs) according to the manufacturer's instructions. Following the cleanup, the mRNA was quantified using the NanoDrop (Thermo Fisher Scientific) and analyzed by agarose gel electrophoresis to confirm the mRNA is the proper size and that no degradation of the mRNA has occurred.

Example 2-2: mRNA encoding influenza A H1N1 virus antigen

The in vitro transcription reaction utilizes a custom mixture of NTPs. The NTPs may comprise chemically modified NTPs, or a mixture of natural and chemically modified NTPs, or natural NTPs. In this Example, 1-methylpseudouridine-5′-triphosphate was used instead of UTP.

A typical in vitro transcription reaction includes the following:
1) Template DNA 1.0 μg
2) 5× transcription buffer 4.0 μl
   (600 mM HEPES-KOH (pH 7.5), 180 mM MgCl₂, 325 mM DTT, 15 mM Spermidine)
3) Bovine serum albumin 2.0 μl
4) NTPs (100 mM each) 8.0 μl
5) RNase inhibitor 20 Units
6) T7 RNA polymerase 100 Units
7) DEPC-treated water up to 20.0 μl

Using the prepared mixture for in vitro transcription reaction, incubation was performed at 37° C. for 2 hours. The crude IVT mixture may be stored at 4° C. overnight for cleanup the next day. The crude IVT mixture was treated with 5 Units of RNase-free DNase I to digest the template DNA. After 30 minutes of incubation at 37° C., the mRNA was purified using Monarch® RNA Cleanup Kit (New England Biolabs) according to the manufacturer's instructions. Following the cleanup, the mRNA was quantified using the NanoDrop (Thermo Fisher Scientific) and analyzed by agarose gel electrophoresis to confirm the mRNA is the proper size and that no degradation of the mRNA has occurred.

One-step capping and 2′-O-methylation capping of uncapped mRNA was performed according to the manufacturers' instructions (New England Biolabs). First, uncapped mRNA and DEPC-treated water were mixed in a final volume of 13.5 μl. The mixture is incubated at 65° C. for 5 minutes to denature mRNA, and then was transferred immediately to ice. The protocol then involves the mixing of 10× capping buffer (2.0 μl); 10mM GTP (1.0 μl); 4 mM S-adenosyl methionine (SAM, 1.0 μl); RNase inhibitor (20 Units); Vaccinia capping enzyme (10 Units); 2′-O-methyltransferase (50 Units) and incubated at 37° C. for 1 hour. The capped mRNA was purified using Monarch® RNA Cleanup Kit (New England Biolabs) according to the manufacturer's instructions. Following the cleanup, the mRNA was quantified using the NanoDrop (Thermo Fisher Scientific) and analyzed by agarose gel electrophoresis to confirm the mRNA is the proper size and that no degradation of the mRNA has occurred.

### Example 3: Preparation of peptide-conjugated, mRNA loaded lipid nanoparticles and vaccine formulation

In this Example, peptide-conjugated, mRNA loaded lipid nanoparticles were manufactured.

Example 3-1: Peptide-conjugated, mRNA loaded nanoparticle enclosing mRNA encoding SARS-COV-2 antigen

In this Example, one mRNA loaded lipid nanoparticle with no surface coating (hereafter S-LNP-0) and three types of peptide-conjugated, mRNA loaded lipid nanoparticles were manufactured: mRNA loaded lipid nanoparticle coated with peptide P-1 (hereafter S-LNP-P-1), mRNA loaded lipid nanoparticle coated with peptide P-2 (hereafter S-LNP-P-2), and mRNA loaded lipid nanoparticle coated with both peptides P-1 and P-2 (hereafter S-LNP-P-1/2). The terminal residue “X” of the SEQ ID NO: 1 and SEQ ID NO: 2 are cysteine because covalent coupling between cysteine of peptide and maleimide attached to the head group of the phospholipid was used in this Example.

S-LNP-0 was manufactured as follows: The S-LNP-0 was prepared by mixing of lipids dissolved in ethanol and mRNA dissolved in 50 mM sodium citrate buffer (pH 4.0) using a NanoAssemblr microfluidic device (Precision Nanosystems). The molar percentage ratio for the constituent lipids was 50% DLin-MC3-DMA (ionizable cationic lipid), 10% DSPC, 38.5% Cholesterol, and 1.5% 1,2-distearoyl-sn-glycero-3-phosphoethanolamine-N-[maleimide(polyethylene glycol)-2000]. At a flow rate ratio of 1:3 ethanol: aqueous phases, the solutions were combined in the microfluidic device. The total combined flow rate was 12 ml/min per microfluidics cartridge. The mixed material was then diluted with phosphate buffered saline (PBS). The diluted particles were dialyzed against PBS (pH 7.2) in Slide-A-Lyzer dialysis cassettes (Thermo Fisher Scientific) for at least 18 hours. The prepared S-LNP-0 was concentrated using Amicon ultra-centrifugal filters (Merck Millipore), and then passed through a 0.22-μm filter and stored at −20° C. until use.

Peptide-conjugated, mRNA loaded lipid nanoparticles S-LNP-P-1 was manufactured as follows: Before peptide coating, mRNA loaded LNPs without peptide coating were manufactured. The LNPs without peptide coating were prepared by mixing of lipids dissolved in ethanol and mRNA dissolved in 50 mM sodium citrate buffer (pH 4.0) using a NanoAssemblr microfluidic device (Precision Nanosystems). The molar percentage ratio for the constituent lipids was 50% DLin-MC3-DMA (ionizable cationic lipid), 10% DSPC, 38.5% Cholesterol, and 1.5% 1,2-distearoyl-sn-glycero-3-phosphoethanolamine-N-[maleimide(polyethylene glycol)-2000]. At a flow rate ratio of 1:3 ethanol:aqueous phases, the solutions were combined in the microfluidic device. The total combined flow rate was 12 ml/min per microfluidics cartridge. The mixed material was then diluted with PBS. The diluted particles were dialyzed against PBS (pH 7.2) in Slide-A-Lyzer dialysis cassettes (Thermo Fisher Scientific) for at least 18 hours. The prepared LNPs were concentrated using Amicon ultra-centrifugal filters (Merck Millipore), and then passed through a 0.22-μm filter and stored at 4° C. (PBS) or −20° C. (20 mM Tris-8% sucrose) until use. After manufacturing of LNPs without peptide coating, peptide coating was performed. Covalent coupling of peptides to LNPs was accomplished under aqueous conditions using a 0.9% NaCl, 10 mM NaHCO₃ aqueous buffer after the LNP preparation described above. For peptide coupling to preformed LNPs, 100 μg of peptide P-1 and 1 μM LNPs were combined in 100 μL of buffer at pH 6.5 at room temperature and were agitated for 8 hours. To remove any unbound peptide, nanoparticles were dialyzed against 1 L of buffer. The dialysis buffer was replaced every hour. The prepared peptide-conjugated, mRNA loaded nanoparticles were stored at −20° C.

Peptide-conjugated, mRNA loaded lipid nanoparticles S-LNP-P-2 was manufactured as follows: Before peptide coating, mRNA loaded LNPs without peptide coating were manufactured. The LNPs without peptide coating were prepared by mixing of lipids dissolved in ethanol and mRNA dissolved in 50 mM sodium citrate buffer (pH 4.0) using a NanoAssemblr microfluidic device (Precision Nanosystems). The molar percentage ratio for the constituent lipids was 50% DLin-MC3-DMA (ionizable cationic lipid), 10% DSPC, 38.5% Cholesterol, and 1.5% 1,2-distearoyl-sn-glycero-3-phosphoethanolamine-N-[maleimide(polyethylene glycol)-2000]. At a flow rate ratio of 1:3 ethanol:aqueous phases, the solutions were combined in the microfluidic device. The total combined flow rate was 12 ml/min per microfluidics cartridge. The mixed material was then diluted with PBS. The diluted particles were dialyzed against PBS (pH 7.2) in Slide-A-Lyzer dialysis cassettes (Thermo Fisher Scientific) for at least 18 hours. The prepared LNPs without peptide coating were concentrated using Amicon ultra-centrifugal filters (Merck Millipore), and then passed through a 0.22-μm filter and stored at 4° C. (PBS) or −20° C. (20 mM Tris-8% sucrose) until use. After manufacturing of LNPs without peptide coating, peptide coating was performed. Covalent coupling of peptides to LNPs was accomplished under aqueous conditions using a 0.9% NaCl, 10 mM NaHCO₃ aqueous buffer after the LNP preparation described above. For peptide coupling to preformed LNPs, 100 μg of peptide P-2 and 1 μM LNPs were combined in 100 μL of buffer at pH 6.5 at room temperature and were agitated for 8 hours. To remove any unbound peptide, nanoparticles were dialyzed against 1 L of buffer. The dialysis buffer was replaced every hour. The prepared peptide-conjugated, mRNA loaded nanoparticles were stored at −20° C.

Peptide-conjugated, mRNA loaded lipid nanoparticles S-LNP-P-1/2 was manufactured as follows: Before peptide coating, mRNA loaded LNPs without peptide coating were manufactured. The LNPs without peptide coating were prepared by mixing of lipids dissolved in ethanol and mRNA dissolved in 50 mM sodium citrate buffer (pH 4.0) using a NanoAssemblr microfluidic device (Precision Nanosystems). The molar percentage ratio for the constituent lipids was 50% DLin-MC3-DMA (ionizable cationic lipid), 10% DSPC, 38.5% Cholesterol, and 1.5% 1,2-distearoyl-sn-glycero-3-phosphoethanolamine-N-[maleimide (polyethylene glycol)-2000]. At a flow rate ratio of 1:3 ethanol:aqueous phases, the solutions were combined in the microfluidic device. The total combined flow rate was 12 ml/min per microfluidics cartridge. The mixed material was then diluted with PBS. The diluted particles were dialyzed against PBS (pH 7.2) in Slide-A-Lyzer dialysis cassettes (Thermo Fisher Scientific) for at least 18 hours. The prepared LNPs without peptide coating were concentrated using Amicon ultra-centrifugal filters (Merck Millipore), and then passed through a 0.22-μm filter and stored at 4° C. (PBS) or −20° C. (20 mM Tris-8% sucrose) until use. After manufacturing of LNPs without peptide coating, peptide coating was performed. Covalent coupling of peptides to LNPs was accomplished under aqueous conditions using a 0.9% NaCl, 10 mM NaHCO₃ aqueous buffer after the LNP preparation described above. For peptide coupling to preformed LNPs, 100 μg of 1:1 mixture of peptide P-1 and peptide P-2 and 1 μM LNPs were combined in 100 μL of buffer at pH 6.5 at room temperature and were agitated for 8 hours. To remove any unbound peptide, nanoparticles were dialyzed against 1 L of buffer. The dialysis buffer was replaced every hour. The prepared peptide-conjugated, mRNA loaded nanoparticles were stored at −20° C.

Example 3-2: Peptide-conjugated, mRNA loaded nanoparticle enclosing mRNA encoding influenza A H1N1 virus antigen

In this Example, one mRNA loaded lipid nanoparticle with no surface coating (hereafter F-LNP-0) and three types of mRNA loaded lipid nanoparticles coated with peptide were manufactured: mRNA loaded lipid nanoparticle coated with peptide P-1 (hereafter F-LNP-P-1), mRNA loaded lipid nanoparticle coated with peptide P-2 (hereafter F-LNP-P-2), and mRNA loaded lipid nanoparticle coated with peptides P-1 and P-2 (hereafter F-LNP-P-1/2). The residue “X” of the SEQ ID NO: 1 and SEQ ID NO: 2 are cysteine because covalent coupling between cysteine of peptide and maleimide attached to the head group of the phospholipid was used in this Example.

F-LNP-0 was manufactured as follows: The F-LNP-0 was prepared by mixing of lipids dissolved in ethanol and mRNA dissolved in 50 mM sodium citrate buffer (pH 4.0) using a NanoAssemblr microfluidic device (Precision Nanosystems). The molar percentage ratio for the constituent lipids was 50% DLin-MC3-DMA (ionizable cationic lipid), 10% DSPC, 38.5% Cholesterol, and 1.5% 1,2-distearoyl-sn-glycero-3-phosphoethanolamine-N-[maleimide (polyethylene glycol)-2000]. At a flow rate ratio of 1:3 ethanol:aqueous phases, the solutions were combined in the microfluidic device. The total combined flow rate was 12 ml/min per microfluidics cartridge. The mixed material was then diluted with PBS. The diluted particles were dialyzed against PBS (pH 7.2) in Slide-A-Lyzer dialysis cassettes (Thermo Fisher Scientific) for at least 18 hours. The prepared F-LNP-0 was concentrated using Amicon ultra-centrifugal filters (Merck Millipore), and then passed through a 0.22-μm filter and stored at −20° C. until use.

Peptide-conjugated, mRNA loaded lipid nanoparticles F-LNP-P-1 was manufactured as follows: Before peptide coating, mRNA loaded LNPs without peptide coating were manufactured. The LNPs without peptide coating were prepared by mixing of lipids dissolved in ethanol and mRNA dissolved in 50 mM sodium citrate buffer (pH 4.0) using a NanoAssemblr microfluidic device (Precision Nanosystems). The molar percentage ratio for the constituent lipids was 50% DLin-MC3-DMA (ionizable cationic lipid), 10% DSPC, 38.5% Cholesterol, and 1.5% 1,2-distearoyl-sn-glycero-3-phosphoethanolamine-N-[maleimide (polyethylene glycol)-2000]. At a flow rate ratio of 1:3 ethanol:aqueous phases, the solutions were combined in the microfluidic device. The total combined flow rate was 12 ml/min per microfluidics cartridge. The mixed material was then diluted with PBS. The diluted particles were dialyzed against PBS (pH 7.2) in Slide-A-Lyzer dialysis cassettes (Thermo Fisher Scientific) for at least 18 hours. The prepared LNPs were concentrated using Amicon ultra-centrifugal filters (Merck Millipore), and then passed through a 0.22-μm filter and stored at 4° C. (PBS) or −20° C. (20 mM Tris-8% sucrose) until use. After manufacturing of LNPs without peptide coating, peptide coating was performed. Covalent coupling of peptides to LNPs was accomplished under aqueous conditions using a 0.9% NaCl, 10 mM NaHCO₃ aqueous buffer after the LNP preparation described above. For peptide coupling to preformed LNPs, 100 μg of peptide P-1 and 1 μM LNPs were combined in 100 μL of buffer at pH 6.5 at room temperature and were agitated for 8 hours. To remove any unbound peptide, nanoparticles were dialyzed against 1 L of buffer. The dialysis buffer was replaced every hour. The prepared peptide-conjugated, mRNA loaded nanoparticles were stored at −20° C.

Peptide-conjugated, mRNA loaded lipid nanoparticles F-LNP-P-2 was manufactured as follows: Before peptide coating, mRNA loaded LNPs without peptide coating were manufactured. The LNPs without peptide coating were prepared by mixing of lipids dissolved in ethanol and mRNA dissolved in 50 mM sodium citrate buffer (pH 4.0) using a NanoAssemblr microfluidic device (Precision Nanosystems). The molar percentage ratio for the constituent lipids was 50% DLin-MC3-DMA (ionizable cationic lipid), 10% DSPC, 38.5% Cholesterol, and 1.5% 1,2-distearoyl-sn-glycero-3-phosphoethanolamine-N-[maleimide (polyethylene glycol)-2000]. At a flow rate ratio of 1:3 ethanol:aqueous phases, the solutions were combined in the microfluidic device. The total combined flow rate was 12 ml/min per microfluidics cartridge. The mixed material was then diluted with PBS. The diluted particles were dialyzed against PBS (pH 7.2) in Slide-A-Lyzer dialysis cassettes (Thermo Fisher Scientific) for at least 18 hours. The prepared LNPs without peptide coating were concentrated using Amicon ultra-centrifugal filters (Merck Millipore), and then passed through a 0.22-μm filter and stored at 4° C. (PBS) or −20° C. (20 mM Tris-8% sucrose) until use. After manufacturing of LNPs without peptide coating, peptide coating was performed. Covalent coupling of peptides to LNPs was accomplished under aqueous conditions using a 0.9% NaCl, 10 mM NaHCO₃ aqueous buffer after the LNP preparation described above. For peptide coupling to preformed LNPs, 100 μg of peptide P-2 and 1 μM LNPs were combined in 100 μL of buffer at pH 6.5 at room temperature and were agitated for 8 hours. To remove any unbound peptide, nanoparticles were dialyzed against 1 L of buffer. The dialysis buffer was replaced every hour. The prepared peptide-conjugated, mRNA loaded nanoparticles were stored at −20° C.

Peptide-conjugated, mRNA loaded lipid nanoparticles F-LNP-P-1/2 was manufactured as follows: Before peptide coating, mRNA loaded LNPs without peptide coating were manufactured. The LNPs without peptide coating were prepared by mixing of lipids dissolved in ethanol and mRNA dissolved in 50 mM sodium citrate buffer (pH 4.0) using a NanoAssemblr microfluidic device (Precision Nanosystems). The molar percentage ratio for the constituent lipids was 50% DLin-MC3-DMA (ionizable cationic lipid), 10% DSPC, 38.5% Cholesterol, and 1.5% 1,2-distearoyl-sn-glycero-3-phosphoethanolamine-N-[maleimide (polyethylene glycol)-2000]. At a flow rate ratio of 1:3 ethanol: aqueous phases, the solutions were combined in the microfluidic device. The total combined flow rate was 12 ml/min per microfluidics cartridge. The mixed material was then diluted with PBS. The diluted particles were dialyzed against PBS (pH 7.2) in Slide-A-Lyzer dialysis cassettes (Thermo Fisher Scientific) for at least 18 hours. The prepared LNPs without peptide coating were concentrated using Amicon ultra-centrifugal filters (Merck Millipore), and then passed through a 0.22-μm filter and stored at 4° C. (PBS) or −20° C. (20 mM Tris-8% sucrose) until use. After manufacturing of LNPs without peptide coating, peptide coating was performed. Covalent coupling of peptides to LNPs was accomplished under aqueous conditions using a 0.9% NaCl, 10 mM NaHCO₃ aqueous buffer after the LNP preparation described above. For peptide coupling to preformed LNPs, 100 μg of 1:1 mixture of peptide P-1 and peptide P-2and 1 μM LNPs were combined in 100 μL of buffer at pH 6.5 at room temperature and were agitated for 8 hours. To remove any unbound peptide, nanoparticles were dialyzed against 1 L of buffer. The dialysis buffer was replaced every hour. The prepared peptide-conjugated, mRNA loaded nanoparticles were stored at −20° C.

### Example 4: Vaccination

Example 4-1: Vaccination with mucosal mRNA vaccine containing mRNA encoding SARS-COV-2 antigen

5-week old female C57Bl/6 mice were used. All mice were housed in a specific pathogen free (SPF) facility. Mice were immunized intranasally with different formulations: (1) the control group G0-CoV was administrated with mucosal mRNA vaccine based on S-LNP-0; (2) the test group G1-CoV was administrated with mucosal mRNA vaccine based on S-LNP-P-1; (3) the test group G2-CoV was administrated with mucosal mRNA vaccine based on S-LNP-P-2; and (4) the test group G3-CoV was administrated with mucosal mRNA vaccine based on S-LNP-P-1/2. Intranasal immunization with mucosal mRNA vaccine containing mRNA encoding SARS-COV-2 antigen was performed at week 0and week 3. Each nasal administration was done as follows: Mice were anesthetized with isoflurane in a gas chamber and queued for nasal administration. Each time a single mouse was taken out of the chamber, held in supine position, nasally administered with mucosal mRNA vaccine (10 μg mRNA) using a P20 pipette (fitted with a gel loading tip) and laid back inside the gas chamber in supine position. This procedure was repeated for the next animal in sequence.

Example 4-2: Vaccination with mucosal mRNA vaccine containing mRNA encoding influenza A H1N1 virus antigen

5-week old female C57Bl/6 mice were used. All mice were housed in an SPF facility. Mice were immunized intranasally with different formulations: (1) the control group G0-Flu was administrated with mucosal mRNA vaccine based on F-LNP-0; (2) the test group G1-Flu was administrated with mucosal mRNA vaccine based on F-LNP-P-1; (3) the test group G2-Flu was administrated with mucosal mRNA vaccine based on F-LNP-P-2; and (4) the test group G3-Flu was administrated with mucosal mRNA vaccine based on F-LNP-P-1/2. Intranasal immunization with mucosal mRNA vaccine containing mRNA encoding influenza A H1N1 virus antigen was performed at week 0 and week 3. Each nasal administration was done as follows: Mice were anesthetized with isoflurane in a gas chamber and queued for nasal administration. Each time a single mouse was taken out of the chamber, held in supine position, nasally administered with mucosal mRNA vaccine (10 μg mRNA) using a P20 pipette (fitted with a gel loading tip) and laid back inside the gas chamber in supine position. This procedure was repeated for the next animal in sequence.

### Example 5: Sample collection and analysis of immune response

Example 5-1: Vaccination with mucosal mRNA vaccine containing mRNA encoding SARS-COV-2 antigen

Sera was collected 15 days after the boost immunization for detection of the humoral response, and nasal wash and bronchoalveolar lavage fluid (BALF) were collected 15 days after the boost immunization for detection of the immune responses including mucosal IgA response. Sera and biological fluids (with protease inhibitors) were kept at −80° C. for long-term storage.

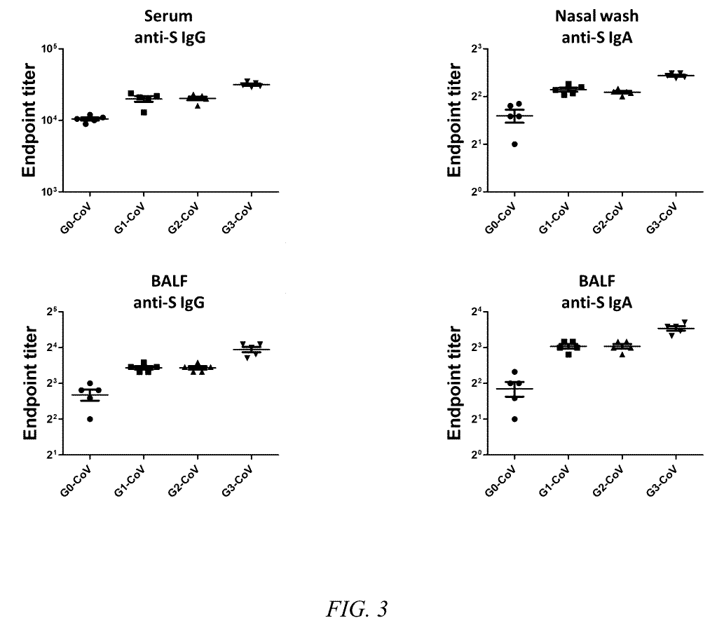

Anti-S protein antibody titers in serum, nasal wash or BALF were determined using ELISA. Briefly, 1 μg/ml spike protein was coated onto ELISA plates in PBS overnight at 4° C. or 2 hours at 37° C. The plate was blocked with PBS+1% BSA+0.1% Tween-20 for 2 hours at room temperature. After washing, the samples were added at different dilutions. The detection was performed using commercially available ELISA kit. For detection, horseradish peroxidase (HRP)-conjugated anti-mouse IgG (1:5,000) and HRP-conjugated anti-mouse IgA (1:10,000) were used. The results are presented in FIG. 3.

These results indicate that intranasal administration of mucosal mRNA vaccine of the present invention induces higher levels of systemic and mucosal responses compared to the conventional mRNA vaccine (the control group).

Example 5-2: Vaccination with mucosal mRNA vaccine containing mRNA encoding influenza A H1N1 virus antigen

Sera was collected 15 days after the boost immunization for detection of the humoral response, and nasal wash and BALF were collected 15 days after the boost immunization for detection of the immune responses including mucosal IgA response. Sera and biological fluids (with protease inhibitors) were kept at −80° C. for long-term storage.

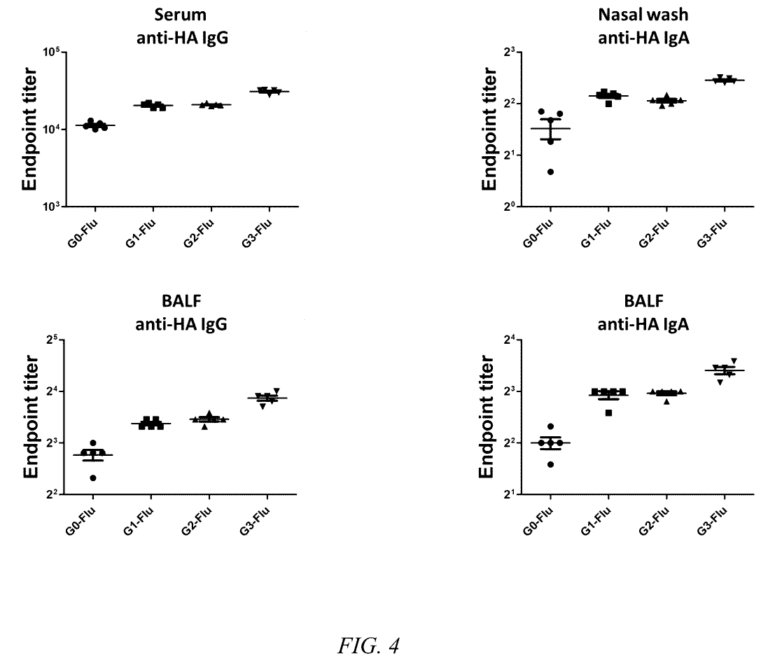

Anti-HA antibody titers in serum, nasal wash or BALF were determined using ELISA. Briefly, 1 μg/ml HA was coated onto ELISA plates in PBS overnight at 4° C. or 2 hours at 37° C. The plate was blocked with PBS+1% BSA+0.1% Tween-20 for 2 hours at room temperature. After washing, the samples were added at different dilutions. The detection was performed using commercially available ELISA kit. For detection, HRP-conjugated anti-mouse IgG (1:5,000) and HRP-conjugated anti-mouse IgA (1:10,000) were used. The results are presented in FIG. 4.

These results indicate that intranasal administration of mucosal mRNA vaccine of the present invention induces higher levels of systemic and mucosal responses compared to the conventional mRNA vaccine (the control group).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A mucosal mRNA vaccine including peptide-conjugated, mRNA loaded nanoparticles coated with at least one peptide selected from the group comprising a peptide having the amino acid sequence as set forth in SEQ ID NO: 1 and a peptide having the amino acid sequence as set forth in SEQ ID NO: 2.

2. The mucosal mRNA vaccine of claim 1, wherein the mRNA codes an antigen derived from bacteria or virus, or an antigen relating to disease.

3. The mucosal mRNA vaccine of claim 1, wherein the nanoparticles are lipid nanoparticles or polymeric nanoparticles.

4. A method for preventing or treating a disease by administration of mucosal mRNA vaccine including peptide-conjugated, mRNA loaded nanoparticles coated with at least one peptide selected from the group comprising a peptide having the amino acid sequence as set forth in SEQ ID NO: 1 and a peptide having the amino acid sequence as set forth in SEQ ID NO: 2.

5. The method for preventing or treating a disease of claim 4, wherein the mRNA codes an antigen derived from bacteria or virus, or an antigen relating to disease.

6. The method for preventing or treating a disease of claim 4, wherein the nanoparticles are lipid nanoparticles or polymeric nanoparticles.

7. A method of preparing mucosal mRNA vaccine including peptide-conjugated, mRNA loaded nanoparticles coated with at least one peptide selected from the group comprising a peptide having the amino acid sequence as set forth in SEQ ID NO: 1 and a peptide having the amino acid sequence as set forth in SEQ ID NO: 2:
preparing mRNA;
forming mRNA loaded nanoparticles; and
conjugating peptide to preformed mRNA loaded nanoparticles.

8. The method of preparing mucosal mRNA vaccine of claim 7, wherein the mRNA codes an antigen derived from bacteria or virus, or an antigen relating to disease.

9. The method of preparing mucosal mRNA vaccine of claim 7, wherein the nanoparticles are lipid nanoparticles or polymeric nanoparticles.
